(19) United States
(12) Patent Application Publication
(10) Pub. No.: US 2010/0297261 A1
(43) Pub. Date: Nov. 25, 2010

(54) Food Product and Fortification System Therefor

(76) Inventors: Salomon Leendert Abrahamse, AT Vlaardingen (NL); van Amelsvoort Netherlands Johannes, AT Vlaardingen (NL); van Buuren Nicodemus M. Bernardus, AT Vlaardingen (NL); Duchateau Servaac M.J.E. Gustaaf, AT Vlaardingen (NL)

Correspondence Address: UNILEVER PATENT GROUP, 800 SYLVAN AVENUE, AG West S. Wing, ENGLEWOOD CLIFFS, NJ, 07632-3100, US

(21) Appl. No.: 11/990,058

(22) PCT Filed: Jul. 18, 2006
(86) PCT No.: PCT/EP2006/007037

§ 371 Date: Feb. 5, 2008

(30) Foreign Application Priority Data
Aug. 15, 2005 (EP) 05076891.0

(51) Int. Cl. A61K 33/26; A23L 1/304; A23L 2/00; A23C 15/00; A23C 13/00; A23G 9/00; A23L 1/40; A23C 9/123; A23L 1/00; A23J 7/00; A23J 1/00; A23D 9/00; A23L 1/30; C07D 307/62

(52) U.S. Cl. 424/646; 426/531; 426/74; 426/590; 426/581; 426/586; 426/565; 426/589; 426/583; 426/618; 426/658; 426/662; 426/656; 426/601; 426/72; 549/315

(57) ABSTRACT

A food product comprising a source of at least one metal ion selected from Fe and Cu ions and an ascorbic acid derivative substituted in the 2-position, wherein the molar ratio of the metal ion or metal ions in the source of metal ions to the ascorbic acid derivative is from 1:0.5 to 1:15.

### FIELD OF THE INVENTION

The present invention relates to a stable food product to combat mineral deficiency (e.g. iron-related anaemia) in mammals based on stable ascorbic acid derivatives to improve mineral bioavailability.

### BACKGROUND OF THE INVENTION

An adequate supply of iron in the diet is essential for preventing iron deficiency with its attendant negative effects on mental, motor and emotional development as well as cognitive performance (1). It is essential that the dietary iron be supplied in a highly bioavailable form. This can be achieved by fortifying foods with e.g. ferrous sulphate and ascorbic acid provided that the ascorbic acid is not lost during storage or meal preparation.

It is well known that ascorbic acid (vitamin C) can improve iron absorption (1, 2), although the mechanism is unclear. It may for example be due to conversion of the redox state of the iron with ascorbic acid operating as an antioxidant, or the formation of a complex between iron and ascorbic acid preventing absorption inhibiting complexation with other food compounds thus enhancing absorption, or via an as yet unexplained mechanism. In all cases the ascorbic acid should be present in its free form and/or be in a form able to react as an antioxidant near the site of absorption.

Combinations of cations with different oxidation levels (such as iron) and ascorbic acid in water-continuous food products result in rapid deterioration of the product as the unsaturated carbon-carbon bonds present in the nutrients are oxidised by a reaction catalysed by ascorbic acid and the cation. Typically, the combination of ascorbic acid and unsaturated lipids (e.g. fatty acids) in a water-containing product will turn rancid in the short term. It is very likely that unsaturated bonds present in other nutrients, micronutrients and vitamins will oxidise as well, rendering the product off taste and/or deactivating the nutritional properties.

It has been reported that in volunteers, iron absorption is better if consumed in encapsulated form in combination with encapsulated ascorbic acid, but worse with non-encapsulated iron, or with encapsulated iron in the absence of ascorbic acid (3). In cases where the diet contains foods with high amounts of phytate or phenolic acids the ratio of ascorbic acid to iron may have to be raised to up to 12:1 by weight to usefully increase iron absorption (4). These findings illustrate the need for the presence of an adequate amount of ascorbic acid relative to the amount of the mineral in a food, during the absorptive phase in the gastro-intestinal tract.

Ascorbic acid derivatives have been developed, which have in common that the ascorbic acid moiety is derivatised on the 2-hydroxyl function, resulting in product stable derivatives as the antioxidant function is blocked. 2-O-α-D-glucopyranosyl-L-ascorbic acid (AA-2G) and ascorbic acid-2-phosphate (AA-2P) are examples of such stable ascorbic acid derivatives. These molecules have been used in diverse applications such as skin care products and in animal feeds.

EP-A 0 947 523 describes acyl derivatives of glycosyl-L-ascorbic acids which have improved oil-solubility. The acyl derivatives are said to release L-ascorbic acid in vivo. The European patent application describes ascorbic acid derivatives that have been substituted in the 2-position and mentions application of these derivatives in food products, cosmetics and pharmaceuticals.

EP-A 0 425 066 describes a crystalline 2-O-α-D-glucopyranosyl-L-ascorbic acid. This crystalline L-ascorbic acid derivative is said to display high stability, to be readily hydrolysable in in vivo and to have satisfactorily high physiological activity. The use of this derivative in foods, beverages, pharmaceuticals and cosmetics is mentioned. It is mentioned that when the crystalline 2-O-α-D-glucopyranosyl-L-ascorbic acid is in free acid form, it can be converted, for example, into sodium salt, magnesium salt, iron salt, copper salt and zinc salt.

EP-A 1 510 140 describes food compositions comprising polyunsaturated fats, 2-substituted ascorbic acid derivates and 5 to 10,000 ppb heave metal ions, such as Fe and Cu ions. The latter ascorbic acid derivatives are said to provide free ascorbic acid in the body when eaten, but to not participate in the regeneration process of the active metal ions. Thus, these ascorbic acid derivatives do not activate the metal ions that catalyse the oxidation reaction between fatty acid and oxygen.

EP-A 0 884 321 describes L-ascorbic acid 2-phosphate zinc salt and a salt hydrate thereof having excellent solubility and exhibiting good stability. The zinc salt does not cause an extreme alkaline condition on dissolving in water and also exhibits antimicrobial activity. The application mentions the use of the zinc salt and salt hydrate in cosmetic and medical preparations, agricultural chemical preparations, animal drug preparations, foodstuff preparations and feedstuff preparations so as to increase the action of vitamin C or to provide an antimicrobial, antiseptic, deodorising or antidandruff effect.

It is known that in guinea pigs, AA-2G can be split during the absorption process by the α-glucosidase in the brush border of the small intestine, resulting in ascorbic acid being absorbed. However, intact AA-2G cannot be absorbed (5). Similarly in humans, AA-2G can be split by the hydrolases in the small intestine resulting in a significant increase in free ascorbic acid in the blood after consumption of AA-2G (6).

### DEFINITION OF THE INVENTION

We have found that ascorbic acid derivatives such as described above can improve iron (or other metal) absorption and bioavailability when incorporated in water-containing food products containing a metal selected from Fe, Cu, whilst avoiding the kind of instability referred to above. An additional advantage is the reduction of negative effects on sensory profile in comparison with conventional products containing iron or copper in combination with ascorbic acid.

Thus, a first aspect of the present invention provides a food product comprising a source of at least one metal ion selected from Fe and Cu ions and an ascorbic acid derivative substituted in the 2-position.

A second aspect of the present invention provides use of an ascorbic acid derivative substituted in the 2-position to increase the bioavailability of one or more metal ions selected from Fe and Cu ions and mixtures thereof, said ascorbic acid derivative and one or more metal ions being present in a food product.

A third aspect of the present invention provides a food product according to the first aspect of the present invention, for preventing or treating iron-deficiency anaemia in a mammal, such as a human.

### DETAILED DESCRIPTION OF THE INVENTION

The ascorbic acid derivative comprises ascorbic acid molecules substituted in the 2-position by a suitable substituent, such as a carbohydrate residue, a polysaccharide residue or an inorganic residue such as a phosphate residue or a sulphate residue. The ascorbic acid derivative with substituent in the 2-position may optionally be in the salt form, e.g. as an alkali (preferably sodium or potassium) salt or alkaline earth metal (preferably magnesium or calcium) salt or a transition metal (preferably iron or zinc) salt.

A particularly preferred substituent in the 2-position is a carbohydrate in the form of a sugar residue or a sugar-containing residue, for example a glucose or fructose residue or a moiety containing glucose or fructose.

The ascorbic acid derivative may, for example, be AA-2G or AA-2P as referred to elsewhere in this specification.

The ascorbic acid derivative substituted in the 2-position may be sourced commercially or prepared by any synthetic route described in any appropriate reference referred to in this specification or in any other reference in any relevant art or by a method analogous to any of these preparative routes as will be readily apparent to persons skilled in the art of synthetic organic chemistry.

Typical inclusion rates of the substituted ascorbic acid in the food product are from 0.001% to 10%, preferably from 0.003% to 6% by weight of the product, based on the equivalent weight of that part of the ascorbic acid derivative molecule(s) corresponding to un-substituted ascorbic acid.

A preferred inclusion rate of the source of said one or more Fe, Cu ions is from 0.001% to 1% by weight of the food product. Especially preferred is an inclusion rate of from 0.002% to 0.005% by weight of the food product. In accordance with another preferred embodiment, the present food product contains more than 0.001%, more preferably at least 0.0015% and most preferably at least 0.002% of the aforementioned metal ions by weight of the food product. Typically, the concentration of said metal ions does not exceed 0.1% by weight of the product. Preferably, the iron cations employed in accordance with present invention are selected from the group consisting of Fe(II) and Fe(III) ions. The copper ions are preferably selected from the group consisting of Cu(I) and Cu(II) ions. According to a particularly preferred embodiment, the metal ions employed in accordance with the present invention are Fe ions.

The molar ratio of the metal ion or metal ions in the source of at least one of Fe and Cu ions to the ascorbic acid derivative is preferably from 1:1 to 1:8, more preferably from 1:2 to 1:4.

A non-limiting description of suitable food products in which the present invention may be imported comprises drinks, including dry mixes to prepare drinks, juices, sports drinks, bars, fat based food products such as spreads, margarines, dressings, mayonnaises, creamers, ice creams, sauces, soups, yoghurts, desserts, toppings, condiments, and bakery, pastry, biscuits and cereal products.

Some typical specific applications in iron-containing products to endow them improved stability (shelf-life) in comparison with those fortified with free ascorbic acid plus iron are:
bar/biscuit; a bar shape product with an outer layer and a filling based on edible fats. The outer layer is composed predominantly from dough made from corn-, wheat- and rice-flour. The filling is a semi-soft solid material made from edible fats, milk powder and sugars. The bar is fortified with vitamins and minerals and formulated in such a way that they will fulfill an optimal nutritional profile.
ready-to-drink formulation is a milk based drink made from milk-powder, edible oils and a mixture of polysaccharides and mono/disaccharides. The drink is fortified with vitamins and minerals and formulated in such a way that they will fulfill an optimal nutritional profile.
porridge/weaning food: a blend of a protein-rich flour (ca 25%) like soybean flour with one or more cereal flours (ca 75%) like maize, millet, sorghum, cassava or wheat, that needs to be cooked for ca 10 minutes before consumption.

When the pH of the food product is from 3.5 to 8.5, it is especially preferred that the ascorbic acid derivative is substituted in the 2-position with a carbohydrate, sulphate or phosphate residue.

In general, any food product according to the present invention may include one or more additional components selected from carbohydrates, for example starches or sugars such as glucose, fructose, maltose, sucrose, as well as peptides, for example soy peptides or casein peptides, fats and oils, for example edible fats and oils, in particular unsaturated oils such as marine or plant seed oils, vitamins other than ascorbic acid, (vitamin C), including fat-soluble vitamins, provitamins, e.g. tocopherols, B-vitamins, carotenoids, fat-soluble anti-oxidants such tocotrienols and emulsifiers, for example lecithin, phospholipids or lysophospholipids.

The present invention will now be explained in more detail by way of the following non-limiting examples.

### Examples

### In Vitro Estimation of Iron Dialyzability

In vitro dializability is an accepted method to estimate mineral (bio)availability (D D Miller et al, Am J Clin Nutr 1981; 34:2248-56; J Luten et al, J Sci Food Agric 1996; 72:415-24). The effect of AA-2G and AA-2P on iron (bio)availability was determined using a dough and under simulation of gastrointestinal conditions (hereinafter referred to as “dissolution experiments”) especially with respect to local hydrolysing enzymes. All glass vessels used were incubated over night in 10% (v/v) HNO₃ in water and subsequently rinsed with demineralised water. Mixing 100 g of “Pelikaan” flour (=wheatflour, Meneba, the Netherlands) and 68 ml of a ferrous sulphate solution resulted in a dough fortified with 50 mg/Kg iron. The dough was divided in portions of 16 g and stored at −20° C. until dissolution experiments took place. Enhancers (AA-2G, AA-2P) were added to the ferrous sulphate solution in a molecular ratio of 1:3 (Fe: enhancer). This resulted in the following:
A dough with 50 mg/Kg iron from ferrous sulphate
A dough with 50 mg/Kg iron from ferrous sulphate and ascorbic acid
A dough with 50 mg/Kg iron from ferrous sulphate and AA-2 G
A dough with 50 mg/Kg iron from ferrous sulphate and AA-2P
A dough with 50 mg/Kg iron from ferrous sulphate; but with ascorbic acid added during dialysis
A dough with 50 mg/Kg iron from ferrous sulphate and AA-2G, with addition of glucosidase enzyme during dialysis
A dough with 50 mg/Kg iron from ferrous sulphate and AA-2P with addition of phosphatase enzyme during dialysis

The dough portions were suspended in water and homogenized for 10 s with a Braun Blender (type 4142) set at maximum speed. Subsequently, water, 0.45 g pepsin and HCl were added to a yield a 90 ml suspension of dough in simulated gastric fluid of pH 2.0. A 20 g sample was taken from the homogenous suspension for determination of total iron. The suspension was transferred to the vessel of a dissolution apparatus (VanK1 VK700, Varian; United States Pharmacopoeia (USP) dissolution apparatus, type II. Conditions for rotations per minute enzyme types and concentrations etc. taken as in the USP; <711> Dissolution; USP26/NF21; 2003; p. 1578-1579). After 120 min incubation at 37° C. and continuous mixing, a sample of the suspension was taken and transferred to an Erlenmeyer flask (a separate sample was taken for the determination of the amount of NaHCO₃ needed to attain pH 7.5). Subsequently, a dialysis bag (Spectra/Por 7 Molecular weight cut-off 8.000) filled with water and a proper amount of NaHCO₃ to adjust the pH of the suspension of dough, pepsin and HCl to pH 7.5 was added to the flask. After 30 min incubation at 37° C. and continuous mixing in a water bath (Lauda MS/2 set at 100 rotations per min), a suspension containing 20 mg pancreatin and 62.5 mg bile-extract was added to the flask. Additional enzyme preparations (glucosidase or phosphatase) were added if appropriate. The resulting suspension was further incubated with the dialysis bag for another 2 hours at 37° C. and continuous mixing. Hereafter, the dialysis bag was removed and a sample of the content of bag was taken to determine the amount of dialyzable iron.

### Measurement of Total Iron Content

Total iron of the wheat flour was determined by inductively coupled plasma atomic emission spectroscopy. Briefly, samples were digested in 5 ml 65% nitric acid and 0.5 ml 30% hydrogen peroxide in closed vessels in a microwave oven at high temperature and high pressure (110 bar). After digestion the volume was adjusted to 50 ml using demineralised water and sprayed into the inductively coupled plasma of the plasma emission spectrometer (Perkin Elmer 3300 DV Inductive Coupled Plasma-Optical Emission Spectrometer). The emission of the individual elements was measured at specific wavelengths and concentrations were quantified from standard solutions.

### Measurement of Iron in Dialysate

Dialyzable iron (sum of Fe²⁺ and Fe³⁺) was determined using a Hitachi 912 Analyser and reagents for the analysis of iron in human serum based on FerroZine (Roche Diagnostics Nederland BV). The analyses were performed according to the instructions of the supplier of the reagents, using the dialysate of the in vitro iron dialyzability assay instead of serum.

### Calculation

Dialyzable iron is expressed as the percentage of the total iron present in the digest, assuming that it had equilibrated across the dialysis membrane by the time the content of the dialysis bag was collected.

### Results

TABLE 1
Total iron content, in vitro dialyzable iron, and relative
iron availability in dough with ferrous sulphate and in
presence of enhancers (AA-2G, AA-2P), as indicated.

| | | In vitro | |
|---|---|---|---|
| | Total iron | dialyzable | |
| | content | Ionic iron | Relative |
| Fortificant | (mg/L) | (% of total iron) | availability * |
| Ferrous sulphate | 49.1 | 3.4 ± 1.8 | 1.0 |
| Ferrous sulphate | 53.0 | 6.1 ± 1.3 | 1.8 |
| with ascorbic acid | | | |
| Ferrous sulphate | 49.1 | 4.6 ± 1.8 | 1.3 |
| with AA-2G | | | |
| Ferrous sulphate | 54.1 | 2.3 ± 1.3 | 0.7 |
| with AA-2P | | | |
| Ferrous sulphate; | 49.1 | 3.9 ± 2.1 | 1.1 |
| ascorbic acid added | | | |
| during dialysis | | | |
| Ferrous sulphate | 49.1 | 8.2 ± 3.9 | 2.4 |
| with AA-2G | | | |
| glucosidase added | | | |
| during dialysis | | | |
| Ferrous sulphate | 54.6 | 2.5 ± 1.3 | 0.7 |
| with AA-2P | | | |
| phosphatase added | | | |
| during dialysis | | | |

Results are means ± SD of 4 or 5 experiments
* Relative to the availability of iron from FeSO₄

The data given in Table 1 show that ascorbic acid increases the amount of available iron about twofold. Adding ascorbic acid only during the dialysis step (intestinal stimulation) does not lead to an increase in available ionic iron. This indicates that the iron needs to be protected from inhibitors (chelated) during the whole experiment. That is, during storage in the product and during consumption up to the time it enters the gastrointestinal tract. AA-2G as such does not give a significant increase of dialyzable iron. Addition of glucosidase enzyme in the dialysis step (intestinal simulation) however leads to a significant increase in available ionic iron. This increase is at least as high as that obtained by using ascorbic acid. The AA-2P derivative does not appear to lead to an increase, which may be due to insufficient phosphatase activity. The current data indicate that the AA-2G derivative of ascorbic acid is a better enhancer than the phosphate form and leads to an increase in available ionic iron compared to ferrous sulphate. AA-2G performs even better than free ascorbic acid when the glucosidase was used in the dialysis step.

### REFERENCES

1. S R Lynch, R J Stoltzfus. Iron and ascorbic acid: proposed fortification levels and recommended iron compounds. J Nutr 2003; 133:2978 S-84S
2. M C Fidler, L Davidsson, C Zeder, T Walczyk, R F Hurrell. Iron absorption from ferrous fumarate in adult women is influenced by ascorbic acid but not by Na₂EDTA. Brit J Nutr 2003; 90:1081-5
3. J B Lee, J Ahn, J Lee, H S Kwak. The microencapsulated ascorbic acid release in vitro and its effect on iron bioavailability. Arch Pharmacol Res 26(10):874
4. O P Garcia, M Diaz, J L Rosado, L H Allen. Ascorbic acid from lime juice does not improve the iron status of iron-deficient women in rural Mexico. Am J Clin Nutr 2003; 78:267-73
5. H. Wakamiya, E Suzuki, I Yamamoto, M Akiba, N Arakawa. In situ intestinal absorption of 2-O-α-D-glucopyranosyl-L-ascorbic acid in guinea pigs. J Nutr Sci Vitaminol 1995; 41:265-72
6. T Oku, S Nakamuru, S Muranaka, S Pgawa, N Sadamori. The bioavailability of ascorbic acid glucoside in humans. Abstr 52^{nd} annual meeting of the Japanese Society of Applied Glycoscience, Sendai, Japan, 24-26 Sep. 2003.

1. A food product comprising a source of at least one metal ion selected from Fe and Cu ions and an ascorbic acid derivative substituted in the 2-position wherein the molar ratio of the metal ion or metal ions in the source of at least one of Fe and Cu ions to the ascorbic acid derivative is from 1:0.5 to 1:15.

2. A food product according to claim 1, wherein the ascorbic acid derivative is substituted in the 2-position by a carbohydrate residue, a phosphate residue or a sulphate residue.

3. A food product according to either preceding claim, wherein the ascorbic acid derivative is substituted in the 2-position by a carbohydrate residue in the form of a sugar residue or a sugar-containing residue such as glucose or fructose or a moiety containing glucose or fructose.

4. A food product according to any preceding claim, comprising from 0.003 to 6 wt % of the ascorbic acid derivative, based on the weight of the ascorbic acid part of the molecule.

5. A food product according to any preceding claim, wherein the amount of its source of at least one of said metal ions is from 0.001% to 1%, preferably from 0.002% to 0.05% by weight of the food product.

6. A food product according to any preceding claim, wherein the food product comprises from 0.002% to 0.05% by weight of a source of at least one of Fe and Cu ions.

7. A food product according to any preceding claim, wherein the molar ratio of the metal ion or metal ions in the source of at least one of Fe and Cu ions to the ascorbic acid derivative is from 1:2 to 1:4.

8. A food product according to any preceding claim, selected from drinks, including dry mixes to prepare drinks, juices, sport drinks, bars, fat based food products such as spreads, margarines, dressings, mayonnaises, creams, ice creams, sauces, soups, yoghurts, desserts, toppings, condiments, bakery, pastry, biscuits and cereal products.

9. A food product according to any preceding claim, wherein the ascorbic acid derivative is a carbohydrate, sulphate or a phosphate, and the pH of the food product is 3.5 to 8.5.

10. A food product according to any preceding claim, further comprising one or more additional ingredients selected from carbohydrates, such as starches or sugars, for example glucose, fructose, maltose, sucrose, peptides, for example soy or casein peptides, fats and oils, for example edible fats and oils, in particular unsaturated oils such as marine or plant seed oils, other vitamins, for example fat-soluble vitamins and provitamins such as tocopherols, B-vitamins and carotenoids, respectively, fat-soluble anti-oxidants such as tocotrienols and emulsifiers, for example lecithin, phospholipids or lysophospholipids.

11. Use of an ascorbic acid derivative substituted in the 2-position to increase the bioavailability of one or more metal ions selected from Fe and Cu ions and mixtures thereof, said ascorbic acid derivative and one or more metal ions being present in a food product.

12. A food product according to any of claims 1 to 10, for treating iron-deficiency anaemia in a mammal.
